(19) United States
(12) Patent Application Publication
(10) Pub. No.: US 2004/0176349 A1
(43) Pub. Date: Sep. 9, 2004

(54) Antibacterial composition

(76) Inventors: Iain Simpson, Cambridge (GB); Hans Rudolph Pfaendler, Stockdorf (DE)

Correspondence Address: MORGAN LEWIS & BOCKIUS LLP, 1111 PENNSYLVANIA AVENUE NW, WASHINGTON, DC, 20004, US

(21) Appl. No.: 10/471,569

(22) PCT Filed: Mar. 14, 2002
(86) PCT No.: PCT/GB02/01162

(30) Foreign Application Priority Data
Mar. 15, 2001 (GB) 1016428.6

(51) Int. Cl. A61K 31/424

(52) U.S. Cl. 514/210.06

(57) ABSTRACT

Disclosed is use of an oxenpenem-3-carboxylic acid of structure (I) or (II) as define above, in combination with an antibiotic and a pharmaceutically acceptable excipient, to prepare a medicament to treat an infection in a human or animal subject caused by a bacterium which does not produce a significant amount of β-lactamase.

This application claims the benefit of U.S. provisional application No. 60/282,166.

### FIELD OF THE INVENTION

This invention relates to antibacterial compositions and methods of preparing antibacterial compositions, and uses thereof.

### BACKGROUND OF THE INVENTION

β-lactam antibiotics are the largest and most successful class of antibacterial agents. There are three major chemical types of β-lactam antibiotic: penicillins; cephalosporins; and carbapenems. All β-lactam antibiotics act by inhibiting enzymes (penicillin binding proteins, “PBPs”) involved in bacterial cell wall synthesis. As with other antibacterial agents, bacterial resistance to β-lactams is an increasingly common problem. The principal mechanism of bacterial resistance to β-lactam antibiotics is the expression of β-lactamases.

β-lactamases are an enormously diverse class of bacterial enzymes which hydrolyse β-lactam antibiotics, rendering them inactive. β-lactamases are encoded by both chromosomal and plasmid-borne genes. Most bacteria, including many of clinical significance, produce at least one chromosomally-encoded β-lactamase. Chromosomally-encoded β-lactamases are bacterial species or sub-species specific and are often inducible. However, many β-lactamases are plasmid-borne and hence can spread rapidly between unrelated bacteria. β-lactamases are classified according to molecular structure:

| | |
|---|---|
| Class A: | Comprises the vast majority of plasmid-mediated β-lactamases |
| Class B: | Comprises zinc requiring metallo β-lactamases |
| Class C: | Comprises the majority of inducible chromosome-mediated |
| | β-lactamases |
| Class D: | Is a small category comprising some plasmid-mediated |
| | β-lactamases. |

Two main strategies to combat β-lactamase mediated resistance have been employed: (1) chemical modification to generate β-lactams with enhanced stability to β-lactamase attack, (2) co-administration of β-lactamase inhibitors.

In relation to (1), chemical modification has resulted in β-lactam antibiotics (particularly cephalosporins and carbapenems) with markedly increased stability to many β-lactamases. However, no β-lactam antibiotic can be described as totally stable to β-lactamase attack. Nevertheless, this process has led to substantial stepwise increases in β-lactamase stability leading to the concept of 1^{st} (e.g. cephaloridine), 2^{nd} (e.g. cefuroxime), 3^{rd} (e.g. ceftazidime) and 4^{th} generation (e.g. cefpirome) cephalosporins.

At present, in relation to (2), there are three β-lactamase inhibitors which are licensed for therapeutic use: clavulanic acid, sulbactam and tazobactam. These inhibitors are used in the following pharmaceutical formulations: (clavulanic acid/amoxycillin e.g. Augmentin™, clavulanic acid/ticarcillin e.g. Timentin™; sulbactam/ampicillin; sulbactam/cefoperazone; and tazobactam/piperacillin e.g. Tazocin™). All three β-lactamase inhibitors are active against Class A β-lactamases, but have little or no usable activity against Class B, C and D β-lactamases. The lack of activity against Class C β-lactamases is a significant clinical problem due to the increasing prevalence of strains producing these enzymes.

A standard test for the production of β-lactamase involves use of the chromogenic cephalosporin, nitrocefin. This compound exhibits a rapid distinctive colour change from yellow (maximum O.D. at pH 7.0 at lamda 390 nm) to red (maximum O.D. at pH 7.0, at lamda 486 nm), as the amide bond in the β-lactam ring is hydrolysed by a β-lactamase (E.C. 3.5.2.6).

Bacteria of the genera Enterococcus and Streptococcus generally do not produce β-lactamases. The genus Enterococcus was formerly classified as part of the genus Streptococcus. The two genera share many characteristics. Enterococcus spp. are Gram positive cocci, non-fastidious organisms which are capable of both aerobic and anaerobic respiration.

Currently there are 15 species recognised. The two most significant species from a clinical viewpoint are E. faecalis and E. faecium, but other species such as E. hirae, E. casseliflavus and E. gallinarum have also been isolated from clinical specimens.

Enterococcus spp. have been associated with urinary tract infections, endocarditis and occasionally severe post-operative septicaemia or septicaemia in immunocompromised patients. Most enterococcal infections are thought to be endogenously acquired, but some cross-infection may occur in hospitalised patients.

Enterococcus spp are considered resistant to cephalosporins, but generally susceptible to penicillins and vancomycin, which are the main chemotherapeutic agents used to combat Enterococcal infections. Oxapenems generally have no clinically relevant activity against Enterococcus spp. However, there is increasing concern at the emergence of vancomycin-resistant enterococci (“VRE”). At least five genes (vanA-vanE) encoding vancomycin resistance have been identified in VRE. Van A and B have been identified in strains of E. faecalis and E. faecium and vanC has been widely observed in E. casseliflavus.

Streptococcus spp. are a large group of Gram-positive cocci, which generally occur in pairs or chains. Streptococci are associated with a range of infections including those of the respiratory tract, skin and soft tissue, meningitis, septicaemia and otitis media. Within the last 10 years, there has been a pandemic increase in resistance to β-lactam antibiotics (so called Penicillin-Resistant Pneumococci—PRP) and there is increasing concern at the rise in resistance to macrolide antibiotics.

Haemophilus spp. are small Gram-negative rods, fastidious in their nutritional requirements and facultative anaerobes. Haemophilus spp. are involved in meningitis, respiratory tract infections, otitis and osteomyelitis. Routine antimicrobial therapy is ampicillin or amoxycillin for β-lactamase negative strains and cefuroxime or a third generation cephalosporin for β-lactamase positive strains.

U.S. Pat. No. 5,108,747 discloses pharmaceutical preparations comprising a β-lactam antibiotic which is normally susceptible to degradation by a β-lactamase, in combination with an oxapenem-3-carboxylic acid having a particular structure. The inventors of U.S. Pat. No. 5,108,747 found that the oxapenem-3-carboxylic acids were stable, and were potent inhibitors of β-lactamase. Accordingly, the person skilled in the art is taught that the pharmaceutical preparations disclosed in U.S. Pat. No. 5,108,747 are effective in treating infections in human or animal subjects caused by β-lactamase producing bacteria.

### SUMMARY OF THE INVENTION

The present inventors have surprisingly found that oxapenem-3-carboxylic acids, of the structure disclosed in U.S. Pat. No. 5,108,747, can greatly increase the efficacy of antibiotics against bacteria which do not produce significant amounts of detectable β-lactamase. Given that oxapenem-3-carboxylic acids are known to be effective β-lactamase inhibitors, but are not considered themselves to have any great amount of antimicrobial activity, this finding was entirely unpredicted.

Accordingly, in a first aspect the invention provides the use of an oxapenem-3-carboxylic acid of structure I or II:

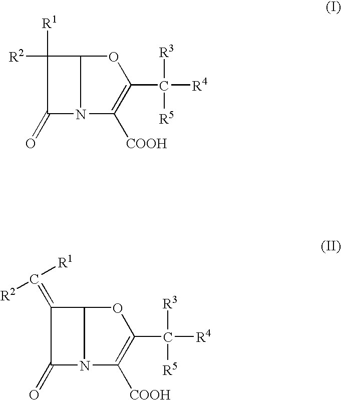

in combination with an antibiotic and a pharmaceutically acceptable excipient, to prepare a medicament to treat an infection in a human or animal (preferably mammalian) subject caused by a bacterium which does not produce a significant amount of β-lactamase, wherein R¹ and R² may, independently, be H, or a pharmaceutically acceptable group comprising from 1 to 10 carbon atoms being connected to the remainder of the molecule by a carbon-carbon single bond; and wherein each of R³, R⁴ and R⁵ is, independently, a pharmaceutically acceptable group comprising from 1 to 10 carbon atoms being connected to the remainder of the molecule by a carbon-carbon single bond.

It will be apparent to those skilled in the art that the carboxylic acid group in the structures I and II may be derivatised to form pharmaceutically acceptable derivatives, such as salts, esters and amides, and such pharmaceutically acceptable derivatives are considered to fall within the scope of the invention. References herein to oxapenem-3-carboxylic acids should therefore be construed, where the context permits, as encompassing derivatives such as salts, esters and amides. Suitable salts include alkali metal, alkaline earth metal salts and primary, secondary or tertiary amine salts.

U.S. Pat. No. 5,108,747, the content of which is incorporated herein by reference, gives extremely detailed teaching as to the permissible and/or desirable properties of structures I and II, and gives detailed teaching as to the methods by which the various oxapenem-3-carboxylic acid compounds may be prepared.

Particularly preferred compounds for use in the present invention are those in which R¹ is H and R² is 1′-hydroxyethyl (H₃CCHOH—). Also preferred are those compounds in which R³ and/or R⁵ are lower alkyl (i.e. methyl, ethyl or propyl). Preferably both R³ and R⁵ are methyl groups.

Examples of two particularly preferred compounds for use in the invention are U* (fill name: (1′R, 5R, 6R) potassium 2-tert-butyl-6-(1′-hydroxyethyl)oxapenem-3-carboxylate) and PFOB (full name: (1′R, 5R, 6R)-2-(4-amino-1,1-dimethylbutyl)-6-(1′-hydroxyethyl) oxapenem-3-carboxylic acid). U* has the structure:

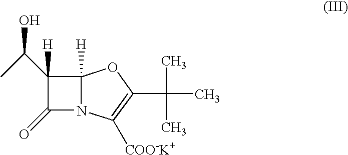

PFOB has the structure:

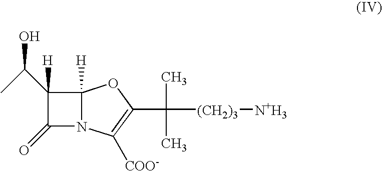

For present purposes, a bacterium may be considered not to produce significant amounts of β-lactamase if, using the “Direct Plate” Method Nitrocefin assay, in accordance with the instructions set out in the 1999 Oxoid manual, a colony of the organism does not turn red within 30 minutes' incubation at room temperature (20° C.) (O'Callaghan et al, 1972 Antimicrob. Ag. & Chemother. 1, 283-288).

The antibiotic employed in the first aspect of the invention defined above may be a naturally-produced antibiotic or may be a synthetic compound. Particularly preferred antibiotics are the β-lactams (penicillins, carbapenems and cephalosporins), and glycopeptides. Examples of suitable antibiotics for use in the medicaments of the invention include amoxycillin, ampicillin, azlocillin, aztreonam, cefazolin, ceftazidime, cefuroxime, cefaclor, cefotaxime, ceftriaxone, ceftizaxime, cefoperazone, cefepime, cefpiroine, cefmenoxime, cefoxitin, cefixime, cefpodoxime, ceftibuten, cefprozil, cephalexin, cephaloridine, imipenem, mecillinam, meropenem, methicillin, moxolactam, panipenem, penicillin G or V, ticarcillin, teicoplanin, and vancomycin. Particularly preferred cephalosporins are ceftazidime (“CAZ”), and cefuroxime.

The inventors have found that medicaments in accordance with the invention are extremely active against bacteria which do not produce β-lactamase, compared to equivalent medicaments which do not comprise an oxapenem β-lactamase inhibitor. This finding was completely unexpected.

The inventors have found that medicaments in accordance with the invention are especially active against bacteria of the genus Enterococcus, and to a lesser extent against Streptococcus spp. and Haemophilus spp., although neither compound is especially active in isolation against these organisms. Thus the invention particularly provides for use of an oxapenem-3-carboxylic acid of structure (I) or (II) and an antibiotic in the preparation of a medicament to treat an Enterococcal infection in a human or animal subject. Typically the animal subject is a mammal, generally a domesticated farm mammal (e.g. horse, pig, cow, sheep, goat etc.) or a companion animal (e.g. cat, dog etc.).

Medicaments in accordance with the invention may be prepared as for conventional pharmaceutical compositions containing antibiotics. Thus, for example, the medicament may be formulated for oral or (preferably) injectable delivery (e.g. intravenous or intramuscular) and may be presented as a capsule, tablet, powder, solution, or suspension. Enteric-coated capsules, which allow for sustained release of capsule contents following oral consumption by a subject are one specific embodiment (although not necessarily a preferred embodiment).

Pharmaceutically acceptable excipients for use in the medicament may include any of those already known in the art, such as gelatin, starch, silica, talc, magnesium stearate, calcium carbonate, sorbitol, glycerol, water, saline and the like. The medicament may optionally comprise conventional additional components, such as binders, stabilizers, preservatives and so on.

The medicaments will generally comprise an oxapenem-3-carboxylic acid of structure (I) or (II) (or derivative thereof, such as a salt, ester or amide) and antibiotic in a ratio of between 1:10 and 10:1, more preferably in a ratio of between 1:10 and 1:1.

The dose of medicament to be delivered will depend at least in part, on the body mass of the subject, the route of delivery, and the severity of the infection. The dose may be, for an average human, in the range of 50-5,000 mg of oxapenem-3-carboxylic acid (with a maximum of about 20 gms per day), with a similar dose of antibiotic. Generally a single dose of medicament will comprise 50-5000 mg of oxapenem-3-carboxylic acid and 50-5000 mg of antibiotic when given by injection, or about 100-5000 mg (typically 250-1000 mg) of each active constituent when given orally.

In a second aspect the invention comprises a method of treating an infection in a human or animal subject caused by bacteria which do not produce significant amounts of β-lactamase; the method comprising administering to the subject an antibiotic and an oxapenem-3-carboxylic acid of the structure I or II.

The two active components may be administered separately by different routes, if desired. Conveniently however the two active agents will be administered by the same route and preferably in a single composition, so as to ensure that they are given contemperaneously to the subject. Typically the method involves the treatment of an Enterococcal infection in the subject, and advantageously will comprise administration of a medicament prepared by the use of the first aspect of the invention defined above. Formulations and dosages will conveniently be used as described previously.

The inventors have further surprisingly found that oxapenem-3-carboxylic acids, according to structure I or II, may exhibit synergistic action when combined with antibiotics which are resistant to β-lactamases, (especially those antibiotics which do not comprise a β-lactam ring structure). Thus in a third aspect the invention provides a pharmaceutical composition for administration to a human or animal subject, the composition comprising: an oxapenem-3-carboxylic acid of structure I or II; an antibiotic which is substantially resistant to degradation by β-lactamase; and a pharmaceutically acceptable excipient. As will be appreciated the composition will generally be sterile and pyrogen-free, when intended for delivery by injection into the subject.

For present purposes, an antibiotic may be considered as substantially resistant to degradation by β-lactamase if the antibiotic retains at least 80% (preferably 90%, more preferably 95%, and most preferably at least 98%) of its initial antimicrobial activity (against an organism which is sensitive to the antibiotic) following incubation for 30 minutes at 37° C. with 1 μg/ml of a β-lactamase at pH 7.0. Percentage antimicrobial activity can be calculated, for example, by determining the reduction in viable count of a sensitive organism following exposure under standard conditions to the antibiotic.

The composition of the third aspect of the invention will typically be formulated for oral or injectable delivery to a mammalian subject, especially a human subject. The composition preferably will be provided in the form of a capsule, tablet, powder, solution or suspension. The composition may be effective against bacteria which produce β-lactamase, and especially effective against bacteria which do not produce significant amounts of β-lactamase (as defined hereinabove).

In a fourth aspect the invention provides a method of treating bacterial infection in a human or animal subject; the method comprising administering to the subject an oxapenem-3-carboxylic acid of structure I or II, and an antibiotic which is substantially resistant to degradation by a β-lactamase.

In a fifth aspect, the invention provides for use of an oxapenem-3-carboxylic acid of structure (I) or (II) defined above, in combination with an antibiotic substantially resistant to degradation by a β-lactamase, and a pharmaceutically acceptable excipient, to prepare a medicament to treat a bacterial infection in a human or animal subject.

The β-lactamase-resistant antibiotic employed in the third, fourth or fifth aspects of the invention may be any suitable antibiotic, especially any suitable non-β-lactam antibiotic, such as glycopeptide and aminoglycoside antibiotics. A specific preferred example is vancomycin and other antibiotics which act on the bacterial cell envelope.

The various aspects of the invention will now be further described by way of illustrative example, and with reference to the accompanying drawings, in which:

FIG. 1A-1C is a schematic illustration of a synthetic route for the compound PFOB;

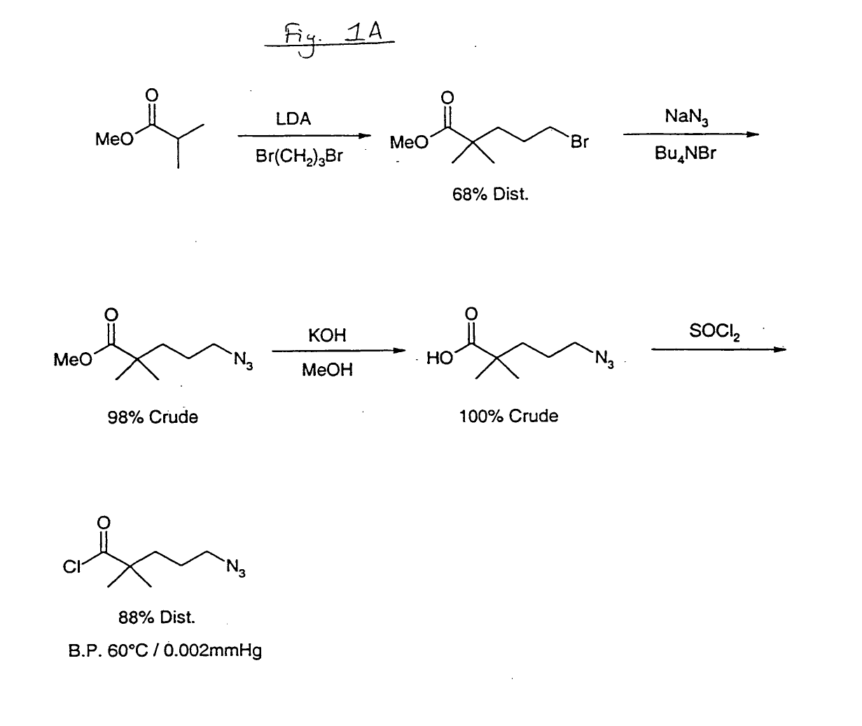

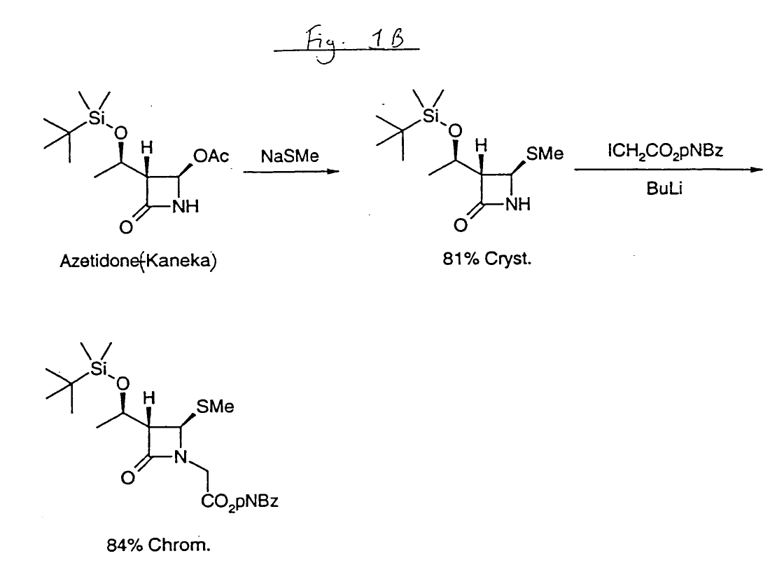

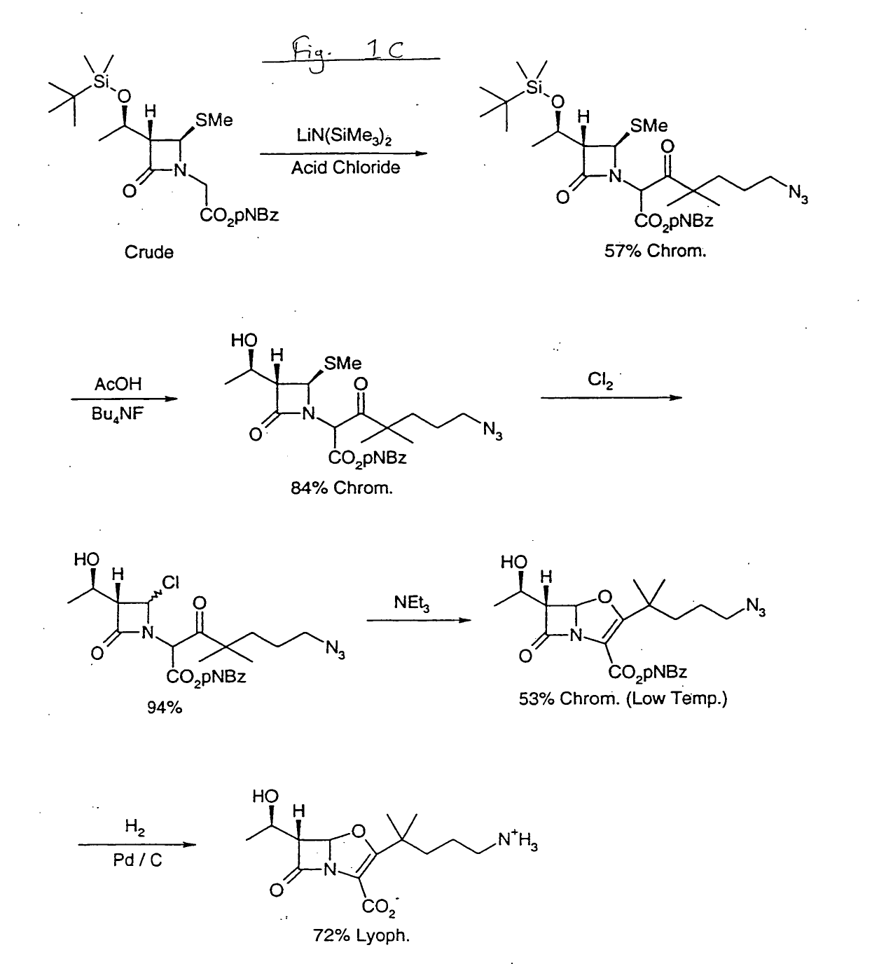

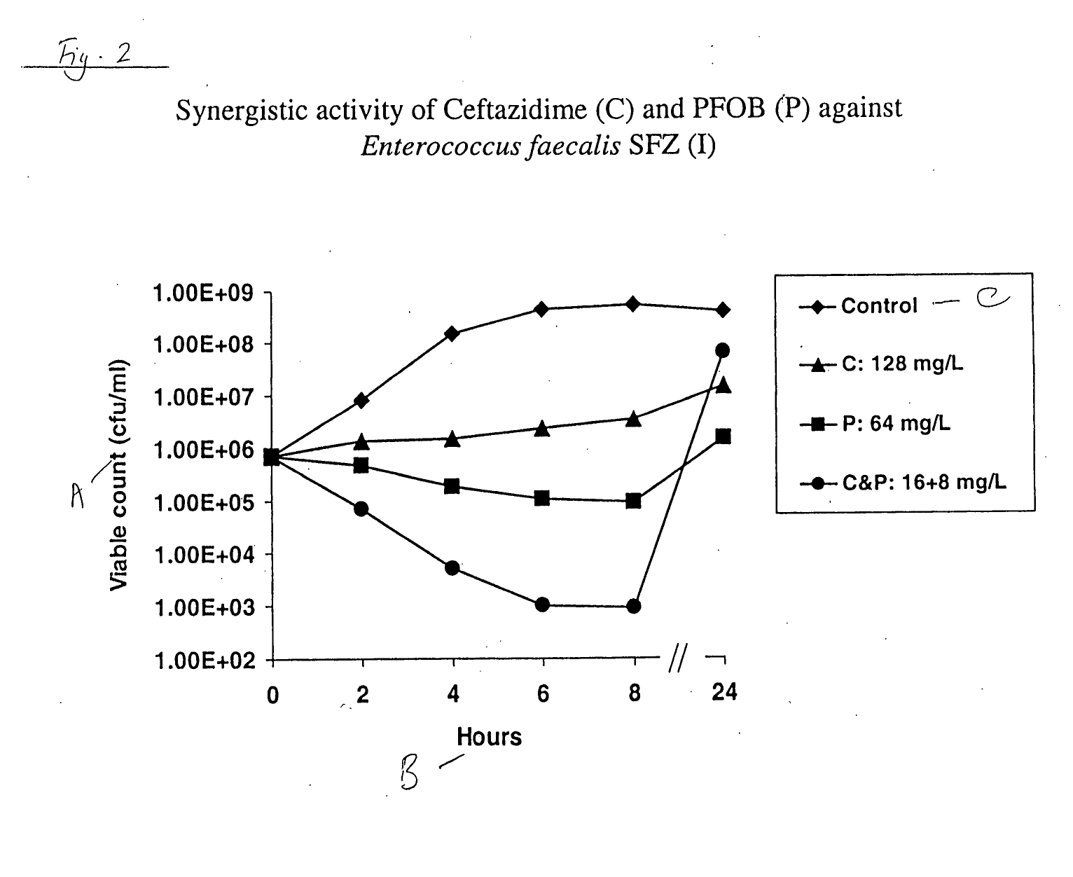

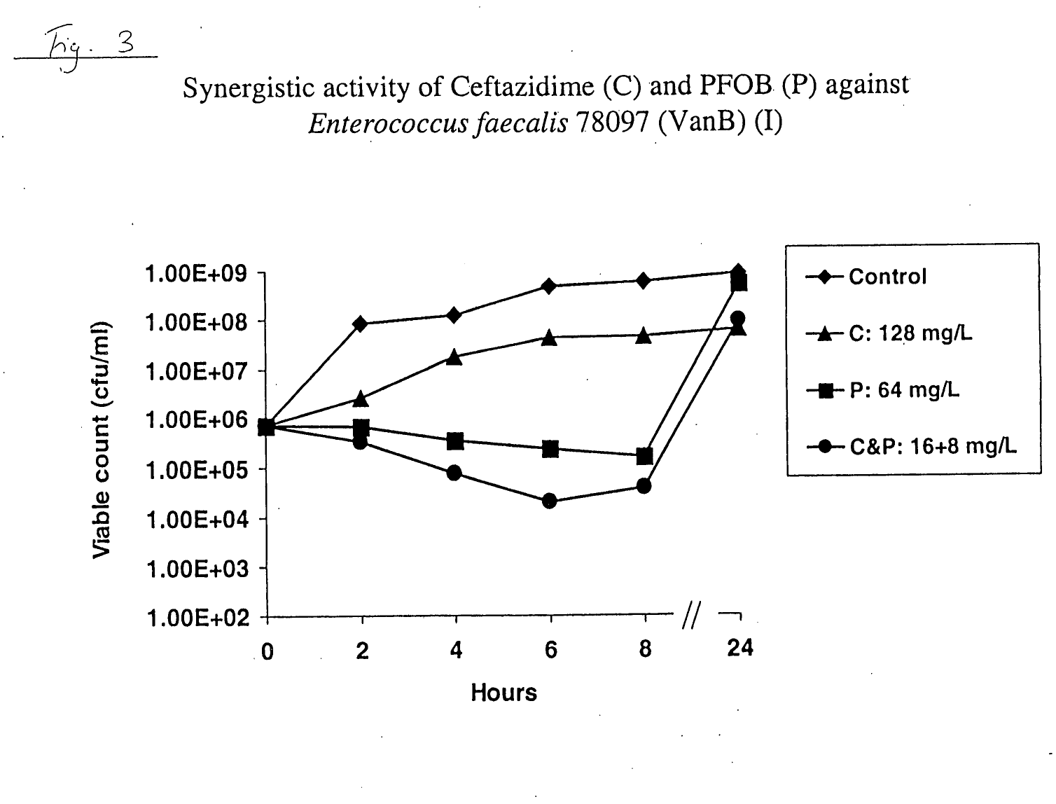

FIGS. 2 and 3 are graphs of viable count (log Cfu/ml) against time (hours) for vancomycin-sensitive and vancomycin-resistant (respectively) isolates of E. faecalis in the presence of various drugs; and

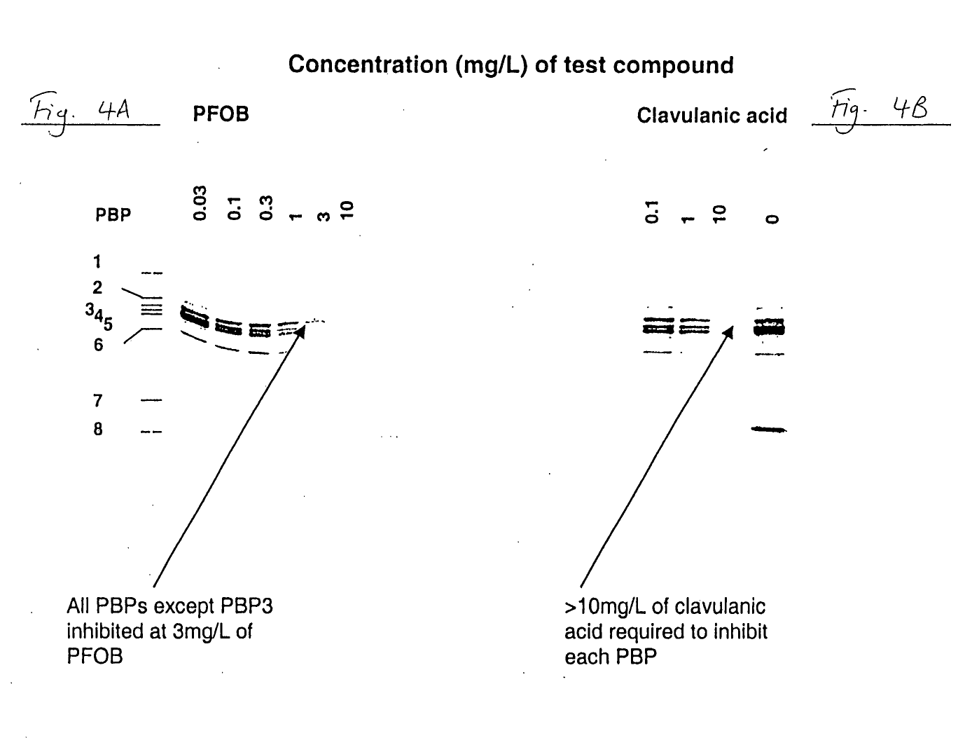

FIGS. 4A and 4B are fluorographs illustrating the affinity of PFOB and clavulanic acid, respectively, for Enterococcal penicillin-binding proteins.

Referring to FIG. 1, FIG. 1A shows the scheme for preparing and acid chloride intermediate; FIG. 1B shows the scheme for preparing a “universal” intermediate starting from a commercially available azetidone (ex. Kaneka); and FIG. 1C shows the final steps of the synthetic route in which the acid chloride (from 1A) and the “universal” intermediate (from 1B) are combined.

### EXAMPLES

### Example 1

In a first experiment, the inventors investigated the activity of three different compounds, in isolation or in particular combinations, against several strains of Enterococcus. (These were generally strains which are publicly available e.g. from the National Collection of Type Cultures or the American Type Culture Collection, and are purely used for the purposes of illustrative example—they are by no means essential for performing the invention.) The compounds tested were the conventional β-lactam cephalosporin antibiotic ceftazidime (“CAZ”), the conventional β-lactamase inhibitor clavulanic acid (“CA”), and an oxapenem-3-carboxylic acid, “PFOB” (compound IV).

The minimum inhibitory concentration (MIC) of the compounds was determined using the microbroth dilution technique in accordance with NCCLS guidelines, employing Mueller-Hinton broth (National Committee for Clinical Laboratory Standards, NCCLS, 1995 “Methods for dilution antimicrobial susceptibility tests for bacteria that grow aerobically”—approved standard M7-A5).

The compounds were tested in isolation, at different concentrations. In addition, the MIC of CAZ was determined when clavulanic acid or PFOB was added at a fixed concentration of 4 mg/L. The results are shown below in Table 1.

Enterococcus spp. are resistant to cephalosporins. Accordingly, as anticipated, CAZ showed no activity against any of the strains tested. CA, being a useful 13-lactamase inhibitor but with no antimicrobial activity in its own right, was similarly inactive against all strains tested, as expected. Likewise PFOB showed no significant activity, in isolation, against any of the Enterococcus strains.

The addition of clavulanic acid at 4 mg/L had no significant effect on the MIC of CAZ. Again, this result was entirely expected, as Enterococcus spp. do not produce significant amounts of β-lactamase and their resistance to CAZ is not β-lactamase mediated, hence inclusion of a β-lactamase inhibitor did not reduce the MIC of CAZ.

However, when CAZ was tested in the presence of PFOB at 4 mg/L, the results obtained were entirely unexpected. The presence of PFOB caused a quite remarkable reduction in the MIC of CAZ against each of the vancomycin-susceptible Enterococci strains, and against one of the vancomycin-resistant strains. This reduction was from a value in excess of 128 mg/L when CAZ was used in isolation, to less than 0.06 mg/L when used in conjunction with PFOB, a reduction of about 2,000 fold. Thus, whilst CAZ is completely ineffective against Enterococcal infections when used in isolation, when used in conjunction with an oxopenem-3-carboxylic acid it has significant clinical potential.

TABLE 1
Activity (MIC mg/L) of PFOB, ceftazidime (CAZ) and clavulanic
acid (CA) alone and in combination against entercocci.

| | | | | | |
|---|---|---|---|---|---|
| | | | | CAZ + | CAZ + |
| | | | | CA @ | PFOB @ |
| Organism | CAZ | CA | PFOB | 4 mg/L | 4 mg/L |

Vancomycin-susceptible strains

| | | | | | |
|---|---|---|---|---|---|
| E. faecalis Phillips | >128 | >128 | 32 | 64 | <0.06 |
| E. faecalis SFZ | >128 | >128 | 64 | 32 | <0.06 |
| E. faecalis NCTC 5957 | >128 | >128 | 32 | 64 | <0.06 |
| E. faecalis 24592 | >128 | >128 | 32 | >128 | <0.06 |
| E. faecium NCTC 7171 | >128 | >128 | 16 | >128 | <0.06 |
| E. hirae ATCC 10541 | >128 | >128 | 16 | >128 | <0.06 |

Vancomycin-resistant strains

| | | | | | |
|---|---|---|---|---|---|
| VRE 300 1562 | >128 | >128 | 128 | >128 | >128 |
| VRE 300 1590 | >128 | >128 | >128 | >128 | >128 |
| VRE 1662 | >128 | >128 | >128 | >128 | >128 |
| VRE 300 2043 | >128 | >128 | 32 | >128 | <0.06 |

### Example 2

The initial findings described in Example 1 above were so promising that the inventors extended their studies to investigate whether PFOB was able to exert a synergistic effect with other antibiotics in addition to ceftazidime.

A “checkerboard” study was conducted, to determine the MIC of various antibiotics (again, using the NCCLS microdilution technique adopted in Example 1) in the presence of various concentrations (0.5-32 mg/L) of PFOB. The aim of the study was to detect any synergy between PFOB and the other antibiotics. For the purposes of the study, synergy was defined in accordance with the FIC (fractional inhibitory concentration) index, as disclosed by Lorian (1991, In “Antibiotics in Laboratory Medicine” 3^{rd} edition, Eds. Williams & Wilkins; esp. p 434-443). In essence, if the combination of test compounds gives at least a 4-fold reduction in the FIC value for each compound, then a synergy may be considered to exist (i.e. FIC of the combination <0.5=synergy; FIC 0.5-2.0=“indifference”; and FIC >2=antagonism).

The results of the study are shown in Table 2. In Table 2, results in the checkerboard indicating synergy are hatched, and specific optimum synergistic combinations are boxed.

The results confirm a marked synergy between PFOB and ceftazidime against vancomycin-susceptible enterococci as exemplified by E. faecalis ATCC 29212 and E. faecium ATCC 10541. Synergy was also observed against E. faecalis 78097, which exhibits vanB-mediated vancomycin resistance.

A less marked, but still significant, synergy was observed between PFOB and any of imipenem, ampicillin and vancomycin. No synergy was observed between PFOB and clavulanic acid.

### Example 3

The kinetics of the antimicrobial activity were further investigated, using the synergistic ceftazidime/PFOB combination, in time-kill experiments against two isolates of E. faecalis: one (E. faecalis SFZ) a vancomycin-sensitive isolate (“VSE”), the other (E. faecalis 78097 van B) a vancomycin-resistant isolate (“VRE”).

Briefly, the experiments were performed as follows: cultures of the VSE and VRE isolates were adjusted to give a final inoculum of approximately 10⁶ cfu/ml in shake flasks containing either no drug (control), ceftazidime at 128 mg/L, PFOB at 64 mg/L or a combination of ceftazidime at 16 mg/L and PFOB at 8 mg/L. Aliquots were removed at hourly intervals, diluted, and viable counts performed. The results are shown in FIGS. 2 & 3. FIG. 2 is a graph of Log viable count (cfu per ml) against time (in hours) for the VSE isolate in the presence or absence of the drugs. FIG. 3 shows the same data for the VRE isolate. The isolates used in the Example are purely illustrative and not especially unusual. Other equivalent strains, publicly available, could equally be employed to demonstrate the efficacy of the invention.

TABLE 2
In vitro activity of ceftazidime, cefuroxime, vancomycin, clavulanic acid, imipenem and ampicillin alone, and in the
presence of varying concentrations of oxapenem PFOB using checkerboard microdilution technique (NCCLS, 1995)

| | MIC (mg/L) or Partner B - lactam antibiotic or | | | | | | | | | | |
|---|---|---|---|---|---|---|---|---|---|---|---|
| Strain | vancomycin in the presence of PFOB at (mg/L): | | | | | | | MIC (alone) of: | | MIC of | |
| Antibiotic partner | 32 | 16 | 8 | 4 | 2 | 1 | 0.5 | PFOB | Partner | PFOB & Partner | Synergy FIC |
| E.faecalis 78097 (van B) | | | | | | | | | | | |
| Ceflazidime | 4 | 8 | 16 | 16 | 32 | >64 | >64 | >32 | >64 | 4 & 16 | 0.156 |
| Cefuroxime | 0.5 | 1 | 4 | 8 | 8 | 8 | 64 | | >64 | 1 & 8 | 0.07 |
| Vancomycin | 16 | 32 | 64 | 64 | 64 | 64 | 64 | | >64 | 16 & 32 | 0.375 |
| Clavulanic acid | >64 | >64 | >64 | >64 | >64 | >64 | >64 | >64 | | | |
| Imipenem | 1 | 1 | 1 | 1 | 1 | 1 | 1 | 0.5 | | | |
| Ampicillin | 0.5 | 0.5 | 0.5 | 0.5 | 0.5 | 0.5 | 0.5 | 1 | | | |
| E. faecalis 5659 (vanA) | | | | | | | | | | | |
| Ceflazidime | 0.25 | >64 | >64 | >64 | >64 | >64 | >64 | >32 | >64 | | |
| Cefuroxime | 0.5 | >64 | >64 | >64 | >64 | >64 | >64 | | >64 | | |
| Vancomycin | >64 | >64 | >64 | >64 | >64 | >64 | >64 | | >64 | | |
| Clavulanic acid | >64 | >64 | >64 | >64 | >64 | >64 | >64 | | >64 | | |
| Imipenem | 0.25 | .05 | 1 | 1 | 1 | 1 | 1 | | 0.25 | | |
| Ampicillin | 0.25 | 1 | 1 | 2 | 2 | 2 | 2 | | 1 | | |
| E. faecalis ATCC 29212 | | | | | | | | | | | |
| Ceflazidime | 2 | 8 | 16 | 32 | >64 | >64 | >64 | >32 | >64 | 8 & 16;4 & 32 | 0.188 |
| Cefuroxime | <0.06 | 1 | 4 | 8 | 32 | >64 | >64 | | 32 | 16 & 1 | 0.156 |
| Vancomycin | 1 | 1 | 2 | 2 | 2 | 2 | 4 | | 4 | 16 & 1 | 0.31 |
| Clavulanic acid | >64 | >64 | >64 | >64 | >64 | >64 | >64 | | >64 | | |
| Imipenem | <0.03 | 0.125 | 0.125 | 0.25 | 0.5 | 0.5 | 0.5 | | 0.5 | 8 & 0.12 | 0.375 |
| Ampicillin | <0.03 | 1 | 1 | 1 | 1 | 1 | 1 | | 0.5 | | |
| E. faecalis ATCC 10541 | | | | | | | | | | | |
| Ceflazidime | <0.06 | 4 | 8 | >64 | >64 | >64 | >64 | 16 | >64 | 8 & 8 | 0.125 |
| Cefuroxime | <0.06 | <0.06 | 0.5 | 8 | >64 | >64 | >64 | | >64 | 8 & 0.5 | 0.067 |
| Vancomycin | 1 | 1 | 2 | 2 | 2 | 2 | 2 | | 4 | | |
| Clavulanic acid | >64 | >64 | >64 | >64 | >64 | >64 | >64 | | >64 | | |
| Imipenem | <0.03 | 0.125 | 0.25 | 1 | 1 | 1 | 1 | | 1 | | |
| Ampicillin | <0.03 | 0.06 | 0.125 | 0.125 | 0.25 | 0.25 | 0.25 | | 1 | | 0.253 |

The legend for both figures is the same i.e. control (no drug)=lozenge symbols; ceftazidime alone at 128 mg/L=triangle symbols; PFOB alone at 64 mg/L=square symbols; and combined ceftazidime (at 16 mg/L) and PFOB (at 8 mg/L)=circular symbols.

FIG. 2 shows that both ceftazidime alone and PFOB alone have some inhibitory effect on growth, but that the combination (bearing in mind the reduced concentration of the drugs and the log scale) is markedly synergistic. FIG. 3 shows qualitatively similar results, although the degree of synergy is not so marked.

It is apparent from both Figures that, after more than 8 hours incubation, the bacterial population recovers and this is presumably due to degradation of the antibacterial drugs. This is of no consequence in practice since, in a clinical setting, the subject would simply be given a repeat dose of the drugs roughly every 8 hours or so, in order to maintain an effective bactericidal concentration.

### Example 4

Further experiments were conducted in an attempt to determine the mechanism of the unexpected synergy between antibiotics (as exemplified by Ceftazidime) and compounds in accordance with general formula I or II (as exemplified by PFOB). The affinity of PFOB for enterococcal penicillin binding proteins (PBPs) was investigated using a conventional radiolabelling assay (Boulton & Orr 1983 “Detection of bacterial penicillin binding proteins and their role in the interpretation of the mode of action of beta-lactam antibiotics” In “Antibiotics: Assessment of antimicrobial activity and resistance” Eds. Denver & Quesnel. Society for Applied Biotechnology, Technical Series 18, pp161-182, Academic Press”). In this assay, the inner bacterial membrane containing the PBPs was extracted, exposed to differing concentrations of test agent (i.e. PFOB) and excess radiolabelled penicillin, followed by separation and visualisation of the PBPs using gel electrophoresis and fluorography.

The results of competition experiments with PFOB, and clavulanic acid are shown in FIGS. 4A and B respectively. In FIG. 4A the test agent (PFOB) is competing with excess radiolabelled penicillin, so in the figures, a weak or missing band indicates that the test agent has successfully competed with penicillin for binding to a PBP, and therefore has a high affinity.

FIG. 4A shows that, at a PFOB concentration of 3 mg/L, nearly all PBPs are bound by PFOB, except PBP3. By comparison with FIG. 4B, it is clear that PFOB has a much higher affinity for PBBs in general than does clavulanic acid.

1. Use of an oxapenem-3-carboxylic acid of structure I or II or a salt, ester or amide derivative thereof: 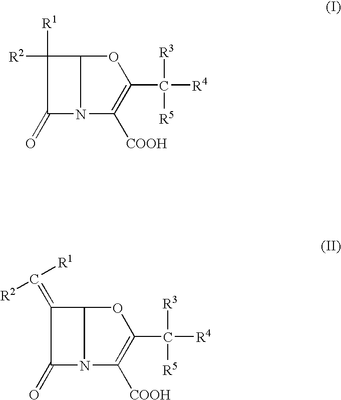
in combination with an antibiotic and a pharmaceutically acceptable excipient, to prepare a medicament to treat an infection in a human or animal (preferably mammalian) subject caused by a bacterium which does not produce a significant amount of β-lactamase, wherein R¹ and R² may, independently, be H, or a pharmaceutically acceptable group comprising from 1 to 10 carbon atoms being connected to the remainder of the molecule by a carbon-carbon single bond; and wherein each of R³, R⁴ and R⁵ is, independently, a pharmaceutically acceptable group comprising from 1 to 10 carbon atoms being connected to the remainder of the molecule by a carbon-carbon single bond.

2. A method of treating an infection in a human or animal subject caused by a bacterium which does not produce a significant amount of a β-lactamase, the method comprising administering to the subject an oxapenem-3-carboxylic acid of structure (I) or (II) as defined above or a salt, ester or amide derivative thereof, and an antibiotic.

3. A use according to claim 1, or a method according to claim 2, wherein the oxapenem-3-carboxylic acid has the structure (III) or (IV) as herein defined.

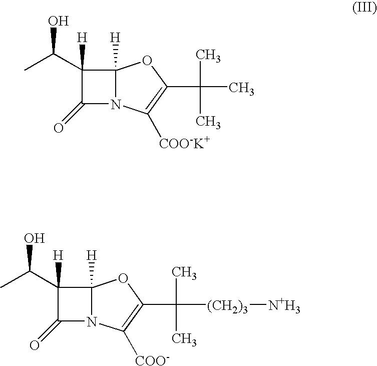

4. A use according to claim 1, or a method according to claim 2, for treatment of an infection caused in a human subject.

5. A use according to claim 1, or a method according to claim 2, for treatment of an infection caused by a bacterium of the genus Enterococcus.

6. A use according to claim 1, or a method according to claim 2, wherein the antibiotic is a cephalosporin.

7. A use according to claim 1, or a method according to claim 2, wherein the antibiotic is ceftazidime or cefuroxime.

8. A pharmaceutical composition for administration to a human or animal subject, comprising: an oxapenem-3-carboxylic acid of structure (I) or (II) defined above or a salt, ester or amide derivative thereof; an antibiotic which is substantially resistant to degradation by a β-lactamase; and a pharmaceutically acceptable excipient.

9. A composition according to claim 8, wherein the antibiotic comprises vancomycin.

10. A composition according to claim 8 or 9, wherein the oxapenem-3-carboxylic acid is in accordance with structure (III) or (IV) defined above.

11. A method of treating a bacterial infection in a human or animal subject, the method comprising administering to the subject an oxapenem-3-carboxylic acid of structure (I) or (II) defined above or a salt, ester or amide derivative thereof, and an antibiotic which is substantially resistant to degradation by a β-lactamase.

12. Use of an oxapenem-3-carboxylic acid of structure (I) or (II) defined above or a salt, ester or amide derivative thereof, in combination with an antibiotic substantially resistant to degradation by a β-lactamase, and a pharmaceutically acceptable excipient, to prepare a medicament to treat a bacterial infection in a human or animal subject.

13. Use of an oxapenem-3-carboxylic acid of structure (I) or (II) defined above or a salt, ester or amide derivative thereof, in combination with an antibiotic and a pharmaceutically acceptable excipient, to prepare a medicament substantially as defined above.

14. A pharmaceutical composition for administration to a human or animal subject, comprising: an oxapenem-3-carboxylic acid of structure (I) or (II) defined above or a salt, ester or amide derivative thereof, in combination with an antibiotic which is substantially resistant to degradation by β-lactamase, and a pharmaceutically acceptable excipient, substantially as defined above.
